(19) United States
(12) Patent Application Publication
(10) Pub. No.: US 2012/0310093 A1
(43) Pub. Date: Dec. 6, 2012

(54) ULTRASOUND IMAGE PRODUCING METHOD AND ULTRASOUND IMAGE DIAGNOSTIC APPARATUS

(75) Inventors: Tsuyoshi TANABE, Kanagawa (JP); Kimito KATSUYAMA, Kanagawa (JP)

(73) Assignee: FUJIFILM CORPORATION, Tokyo (JP)

(21) Appl. No.: 13/484,306

(22) Filed: May 31, 2012

(30) Foreign Application Priority Data
Jun. 6, 2011 (JP) 2011-126173
Jun. 6, 2011 (JP) 2011-126191

(51) Int. Cl. A61B 8/14

(52) U.S. Cl. 600/443

(57) ABSTRACT

Provided are an ultrasound image producing method and an ultrasound image diagnostic apparatus that can quickly decide the presence or the absence of influence of refraction, can shorten the time taken for measurement or calculation, can perform measurement or calculation with a small error, and can calculate accurate local sound velocity values. A lattice is set in a region-of-interest, environmental sound velocity values of two or more lattice points located at different positions in a scanning direction of ultrasonic waves are measured as preliminary sound velocity measurement, and main sound velocity measurement that calculates local sound velocity values in lattice points is performed when the difference between a maximum value and a minimum value of the measured environmental sound velocity values is equal to or lower than a predetermined threshold.

### BACKGROUND OF THE INVENTION

The present invention relates to an ultrasound image producing method and an ultrasound image diagnostic apparatus for performing imaging of internal organs or the like within a living body by transmission and reception of ultrasonic waves and producing an ultrasound diagnostic image thereof to be used for diagnosis.

Hitherto, ultrasound diagnostic apparatuses using an ultrasound image have been put into practical use in the medical field. Generally, this type of ultrasound diagnostic apparatus has an ultrasound probe in which a transducer array is built, and an apparatus body connected to the ultrasound probe. Ultrasonic waves are transmitted toward a subject from the ultrasound probe, ultrasonic echoes from the subject are received and converted to reception signals by the ultrasound probe, and the reception signals are electrically processed in the apparatus body to produce an ultrasound image.

In the ultrasound diagnostic apparatus, the ultrasound image is produced assuming that the sound velocity within the living body of a subject is constant. However, since there is variation in actual sound velocity values within the living body, a spatial distortion occurs in the ultrasound image due to the variation.

In this regard, in recent years, in order to diagnose a part within a subject with higher precision, sound velocity values (local sound velocity values) in arbitrary diagnosed part are measured, and such distortion of image is corrected.

For example, JP 2010-99452 A suggests an ultrasound diagnostic apparatus that sets a plurality of lattice points around a diagnosed part, measures environmental sound velocity values (optimal sound velocity values) on the basis of reception data obtained by transmitting and receiving ultrasonic beams to/from the respective lattice points, and calculates local sound velocity values in the respective lattice points from the environmental sound velocity values of the plurality of lattice points.

Also, JP 2009-279306 A suggests an ultrasound diagnostic apparatus that decides the degree of beam convergence in focus processing in a plurality of first regions, calculates sound velocity values in the respective regions, and calculates sound velocity values in a plurality of second regions that are subdivided more than the first regions.

### SUMMARY OF THE INVENTION

However, when the sound velocity values within the living body are measured, the sound velocity values (environmental sound velocity values) acquired by transmission and reception of ultrasonic beams may be disturbed in the azimuth direction (longitudinal direction). For example, when the abdomen is measured, sound velocity values are disturbed in the orientation direction. It is presumed that sound waves are refracted in a fat layer or muscular layer of the abdominal wall before reaching the liver. As such, in a certain region, sound velocity values may be measured so as to be higher than actual sound velocities due to the influence of refraction or the like.

When the local sound velocity values in the respective lattice points are calculated from the environmental sound velocity values of the plurality of lattice points as in JP 2010-99452 A, or when sound velocity values are obtained in the plurality of subdivided second regions as in JP 2009-279306 A, if the environmental sound velocities (sound velocity values of the first regions) are measured to be higher than the actual sound velocities, there is a concern that accurate local sound velocity values (sound velocity values of the second regions) cannot be calculated.

Additionally, when the local sound velocity values are obtained as in JP 2010-99452 A or JP 2009-279306 A, substantial calculation time is taken. Thus, calculation time is wastefully taken in the case where the measurement error due to the influence of refraction is identified after all measurements or calculations are performed.

An object of the invention is to solve the problems in the above related art and to provide an ultrasound image producing method and an ultrasound image diagnostic apparatus that can quickly grasp the presence or the absence of the influence of refraction, can shorten the time taken for measurement or calculation, and also can perform measurement or calculation with a small error thereby obtaining accurate local sound velocity values.

In order to achieve the above objects, the present invention provides an ultrasound image producing method comprising: an image producing step for transmitting ultrasonic waves and receiving ultrasonic echoes reflected by a subject to output reception signals according to the received ultrasonic waves, by a transducer array, and performing signal processing of the reception signals to produce a B-mode image by an image producer; a lattice setting step for setting a region-of-interest on the produced B-mode image and setting a lattice on the set region-of-interest; a preliminary sound velocity measuring step for measuring environmental sound velocity values of two or more lattice points of the lattice located at different positions in a scanning direction of the ultrasonic waves; a detecting step for detecting whether or not a measured sound velocity difference that is a difference between a maximum value and a minimum value in environmental sound velocity values measured in the preliminary sound velocity measuring step is equal to or lower than a predetermined threshold; and a main sound velocity measuring step for calculating local sound velocity values in lattice points of the lattice when the measured sound velocity difference is equal to or lower than the predetermined threshold.

Preferably, environmental sound velocity values of two or more lattice points in the scanning direction of the ultrasonic waves are further measured at different depths in the preliminary sound velocity measuring step, and whether or not measured sound velocity differences are equal to or lower than predetermined threshold at all the depths is detected in the detecting step, and the main sound velocity measuring step is performed when the measured sound velocity differences are equal to or lower than the predetermined threshold at all the depths.

A lattice with a size that exceeds the set region-of-interest is preferably set in the lattice setting step.

A number of lattice points where environmental sound velocity values are to be measured in the preliminary sound velocity measuring step is preferably smaller than a number of lattice points where local sound velocity values are to be calculated in the main sound velocity measuring step.

Preferably, the ultrasound image producing method further comprises a step for comparing the environmental sound velocity values measured in the preliminary sound velocity measuring step and displaying a lattice point showing a largest environmental sound velocity value in a discriminative manner.

Preferably, the ultrasound image producing method further comprises a step for displaying notice or measurement results of environmental sound velocity values when the difference between the maximum value and the minimum value in the environmental sound velocity values exceeds the predetermined threshold in the detecting step.

Measurement results of the local sound velocity values in the main sound velocity measuring step are preferably superimposed and displayed on the B-mode image.

Preferably, the transducer array is a two-dimensional transducer array having transducers that are arranged two-dimensionally, and acquiring a plurality of information on two-dimensional tomographic planes in a direction orthogonal to the tomographic planes, according to an arrangement of the transducers, the preliminary sound velocity measuring step and the detecting step are performed in each of the plurality of tomographic planes, and the main sound velocity measuring step is performed in a tomographic plane where the measured sound velocity difference is equal to or lower than the predetermined threshold.

Preferably, the ultrasound image producing method further comprises a step for selecting a tomographic plane where the measured sound velocity difference is a smallest, and the main sound velocity measuring step is performed in the selected tomographic plane.

Preferably, the preliminary sound velocity measuring step and the detecting step are performed sequentially in the plurality of tomographic planes, and the main sound velocity measuring step is performed in a tomographic plane where the measured sound velocity difference becomes equal to or lower than the predetermined threshold first.

In order to achieve the above objects, the present invention also provides an ultrasound image diagnostic apparatus comprising: a transducer array that transmits ultrasonic waves and receives ultrasonic echoes reflected by a subject to output reception signals according to the received ultrasonic waves; an image producer that produces an ultrasound image based on the reception signals output from the transducer array; a region-of-interest setter that sets a region-of-interest within an imaging region, sets a lattice on the set region-of-interest, and sets lattice points; a preliminary sound velocity measuring unit that measures environmental sound velocity values of two or more lattice points of the lattice located at different positions in a scanning direction of the ultrasonic waves, and that calculates a measured sound velocity difference that is a difference between a maximum value and a minimum value in the measured environmental sound velocity values; and a main sound velocity measuring unit that calculates local sound velocity values in lattice points of the lattice when the measured sound velocity difference calculated by the preliminary sound velocity measuring unit is equal to or lower than the predetermined value.

Preferably, the transducer array is a two-dimensional transducer array having transducers that are arranged two-dimensionally, and acquiring a plurality of information on two-dimensional tomographic planes in a direction orthogonal to the tomographic planes, according to an arrangement direction of the transducers, the preliminary sound velocity measuring unit calculates the measured sound velocity differences in each of the tomographic planes, and the main sound velocity measuring unit calculates local sound velocity values in lattice points of the lattice, in a tomographic plane where the measured sound velocity difference calculated by the preliminary sound velocity measuring unit is equal to or lower than the predetermined threshold.

According to the ultrasound image producing method and the ultrasound image diagnostic apparatus of the invention having the above configuration, environmental sound velocity values of two or more lattice points that are different in the scanning direction of ultrasonic waves in lattice are respectively measured as the preliminary sound velocity measurement before the local sound velocity values are calculated, and the main sound velocity measurement is performed when the difference between a maximum value and a minimum value of the measured environmental sound velocity values is equal to or lower than a predetermined threshold. Thus, the presence or the absence of the influence of refraction can be grasped quickly, the time taken for measurement or calculation can be shortened, and also measurement or calculation can be performed with a small error thereby obtaining accurate local sound velocity values.

### BRIEF DESCRIPTION OF THE DRAWINGS

FIG. 1 is a block diagram conceptually illustrating the configuration of an ultrasound diagnostic apparatus that implements an ultrasound image producing method of the invention.

FIG. 2 is a block diagram conceptually illustrating the configuration of the sound-velocity calculator shown in FIG. 1.

FIG. 3 is a view schematically illustrating set lattice points.

FIGS. 4A and 4B are views schematically illustrating selected lattice points for preliminary measurement.

FIGS. 5A and 5B are views schematically illustrating measurement results of environmental sound velocity values.

FIG. 6 is a view schematically illustrating lattice points.

FIGS. 7A and 7B are views schematically illustrating the principle of sound velocity calculation.

FIG. 8 is a flowchart for explaining the operation of the ultrasound diagnostic apparatus of FIG. 1.

FIG. 9 is a view schematically illustrating lattice points for preliminary measurement.

FIG. 10 is a block diagram conceptually illustrating the configuration of another example of the ultrasound diagnostic apparatus that implements the ultrasound image producing method of the invention.

FIG. 11 is a view schematically illustrating a two-dimensional transducer array and tomographic planes.

FIG. 12 is a block diagram conceptually illustrating the configuration of the sound-velocity calculator shown in FIG. 10.

FIGS. 13A and 13B are views schematically illustrating selected lattice points for preliminary measurement.

FIGS. 14A and 145 are views schematically illustrating measurement results of environmental sound velocity values.

FIG. 15 is a flowchart for explaining the operation of the ultrasound diagnostic apparatus of FIG. 10.

FIG. 16 is a view schematically illustrating lattice points for preliminary measurement.

### DETAILED DESCRIPTION OF THE INVENTION

An ultrasound diagnostic apparatus that implements an ultrasound image producing method of the invention will be described below in detail on the basis of preferred embodiments illustrated in the accompanying drawings.

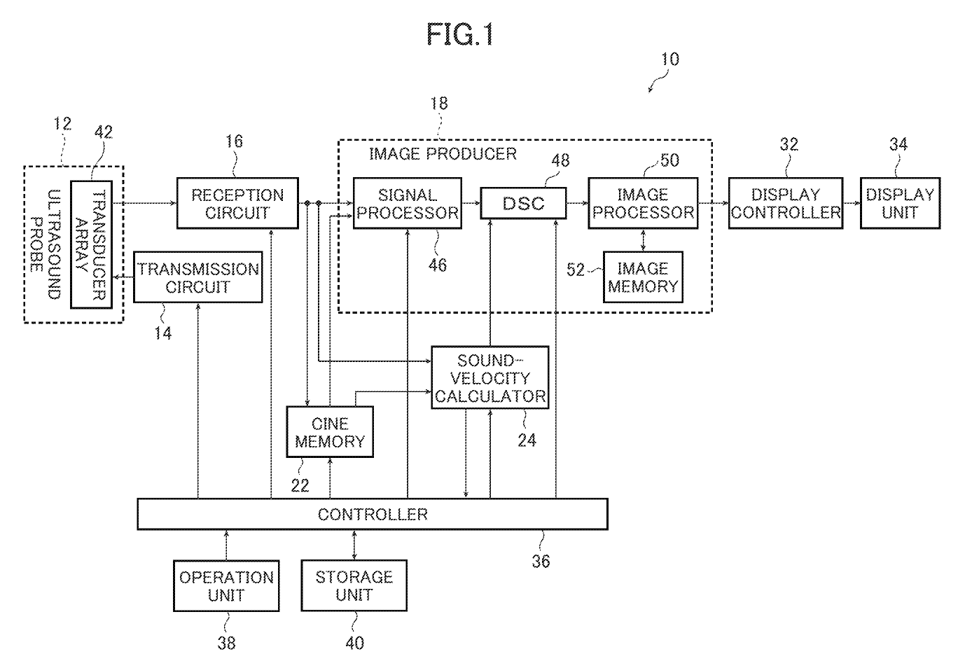

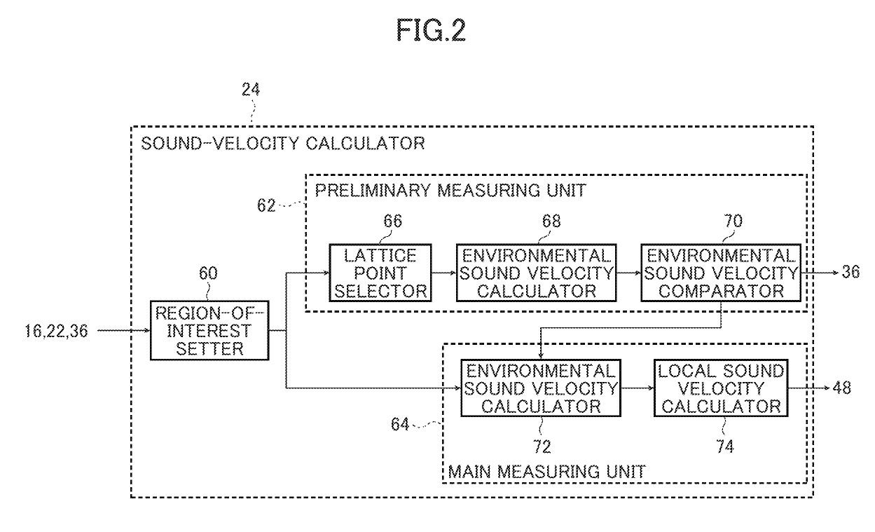

FIG. 1 is a block diagram conceptually illustrating the configuration of one example of the ultrasound diagnostic apparatus that implements the ultrasound image producing method of the invention, and FIG. 2 is a block diagram conceptually illustrating the configuration of a sound-velocity calculator 24.

The ultrasound diagnostic apparatus 10 has an ultrasound probe 12, a transmission circuit 14 and a reception circuit 16 that are connected to the ultrasound probe 12, an image producer 18, a cine memory 22, a sound-velocity calculator 24, a display controller 32, a display unit 34, a controller 36, an operation unit 38, and a storage unit 40.

The ultrasound probe 12 has a transducer array 42 used for a usual ultrasound diagnostic apparatus.

The transducer array 42 has a plurality of ultrasound transducers that are arranged one-dimensionally or two dimensionally. In the imaging of the ultrasound image, the ultrasound transducers transmit ultrasonic beams according to actuation signals supplied from the transmission circuit 14, and receive ultrasonic echoes from a subject to output reception signals.

Each of the ultrasound transducers comprises a vibrator composed of a piezoelectric body and electrodes each provided on both ends of the piezoelectric body. The piezoelectric body is composed of, for example, a piezoelectric ceramic represented by a PZT (lead zirconate titanate), a polymeric piezoelectric device represented by PVDF (polyvinylidene fluoride), or a piezoelectric monocrystal represented by PMN-PT (lead magnesium niobate lead titanate solid solution).

If a pulsed or continuous-wave voltage is applied to the electrodes of the vibrators, the piezoelectric substances expand and contract, pulsed or continuous-wave ultrasonic waves are generated from the respective vibrators, and an ultrasonic beam is formed by the synthesis of the ultrasonic waves. If the respective vibrators receive propagated ultrasonic waves, they expand and contract to generate electrical signals, and the electrical signals are output as reception signals of the ultrasonic waves.

The transmission circuit 14 includes, for example, a plurality of pulsers, and on the basis of a transmission delay pattern selected according to a control signal from the controller 36, the transmission circuit 14 adjusts the delay amounts of the respective actuation signals such that the ultrasonic waves transmitted from the plurality of ultrasound transducers of the transducer array 42 form an ultrasonic beam and supplies the adjusted actuation signals to the plurality of ultrasound transducers.

The reception circuit 16 performs amplification and A/D conversion of the reception signals transmitted from the respective ultrasound transducers of the transducer array 42, and then, performs reception focus processing by allotting and adding the respective delays to the respective reception signals, according to the sound velocity or the distribution of the sound velocity set based on a reception delay pattern selected according to a control signal from the controller 36. Reception data (sound ray signals) in which the foci of ultrasonic echoes are narrowed are produced by this reception focusing processing.

The reception circuit 16 supplies the reception data to the image producer 18, the cine memory 22, and the sound-velocity calculator 24.

The image producer 18 produces an ultrasound image based on the reception data supplied from the reception circuit 16.

The image producer 18 has a signal processor 46, a DSC 48, an image processor 50, and an image memory 52.

The signal processor 46 performs the correction of attenuation based on distance, on the reception data produced by the reception circuit 16, according to the depth of the reflection positions of the ultrasonic waves, and then performs envelope detection processing thereby producing B-mode image signals that are tomographic image information regarding tissue within a subject.

The DSC (Digital Scan Converter) 48 converts (raster-converts) the B-mode image signals produced in the signal processor 46 into image signals according to a normal television signal scan system.

The DSC 48 also converts signals of a sound velocity map, supplied from the sound-velocity calculator 24 to be described below, into image signals according to the normal television signal scan system.

The image processor 50 performs various kinds of required image processing, such as gradation processing, on the B-mode image signals input from the DSC 48, and then, outputs the processed B-mode image signals to the display controller 32 or stores these signals in the image memory 52.

The display controller 32 makes an ultrasound diagnostic image display on the display unit 34 on the basis of the B-mode image signals subjected to the image processing by the image processor 50.

The display unit 34 includes a display device, for example, such as an LCD, and displays the ultrasound diagnostic image under the control of the display controller 32.

The cine memory 22 sequentially stores the reception data output from the reception circuit 16. The cine memory 22 also stores information on the frame rate (for example, the depth of the reflection positions of ultrasonic waves, the density of scanning lines, and the parameters representing visual field widths) input from the controller 36 in association with the above reception data.

The sound-velocity calculator 24 is the part that calculates local sound velocity values in tissue to be diagnosed in a subject and produces a sound velocity map showing sound velocity values and position information, under the control of the controller 36.

Here, in the invention, the sound-velocity calculator 24 performs preliminary sound velocity measurement that measures environmental sound velocity values to decide the presence or the absence of a measurement error caused by refraction, and then, performs main sound velocity measurement that measures the local sound velocity values.

The sound-velocity calculator 24 has a region-of-interest setter 60, a preliminary measuring unit 62, and a main measuring unit 64.

The region-of-interest setter 60 sets a region-of-interest ROI within a subject, sets a lattice two-dimensionally in the region-of-interest ROI, and sets two-dimensionally in the depth direction and the azimuth direction (scanning direction of ultrasonic waves) a plurality of lattice points X_{ROI} subjected to the measurement.

The region-of-interest setter 60 sets the region-of-interest ROI according to the instruction input from the operation unit 38 by an operator.

The region-of-interest setter 60 also sets a plurality of lattice points X_{ROI} (lattice) according to the set region-of-interest ROI.

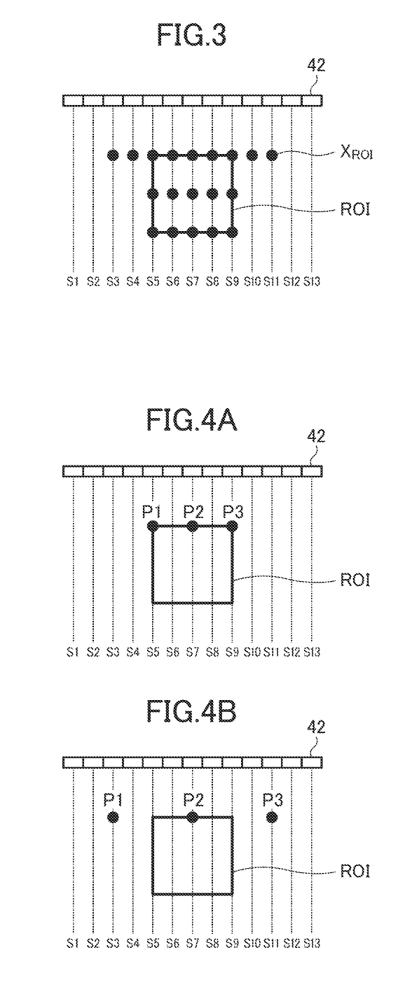

FIG. 3 is a view schematically illustrating the region-of-interest ROI and the lattice points X_{ROI} that are set.

In FIG. 3, broken lines S1 to S13 conceptually show sound rays of an ultrasonic beam transmitted from the transducer array 42. As shown in FIG. 3, the lattice points X_{ROI} are set for every sound ray within the region-of-interest ROI in the azimuth direction (scanning direction of ultrasonic waves). At shallow positions in the depth direction, the lattice points X_{ROI} are also set outside the region-of-interest ROI.

In the illustrated example, three lattice points X_{R01} are set in the depth direction. However, the lattice points are not limited to this, and a plurality of points are set according to resolving power, processing time, or the like.

The region-of-interest setter 60 supplies information on the region-of-interest ROI and the plurality of lattice points X_{ROI} that are set to the preliminary measuring unit 62 and the main measuring unit 64.

As preliminary measurement before the measurement (main sound velocity measurement) of local sound velocity values, the preliminary measuring unit 62 measures environmental sound velocity values regarding several points among the plurality of lattice points X_{ROI} set by the region-of-interest setter 60 and decides the presence or the absence of a measurement error caused by refraction.

The preliminary measuring unit 62 has a lattice point selector 66, an environmental sound velocity calculator 68, and an environmental sound velocity comparator 70.

The lattice point selector 66 selects lattice points to be used for the preliminary measurement from the plurality of lattice points X_{ROI} set by the region-of-interest setter 60. Here, the number of the lattice points for preliminary measurement selected by the lattice point selector 66 is smaller than the number of lattice points subjected to the measurement of local sound velocity values in the main sound velocity measurement.

FIG. 4A is a view schematically illustrating selected lattice points for preliminary measurement.

As shown in FIG. 4A, the lattice point selector 66 selects lattice points P1 and P3 located at both ends of the region-of-interest ROI in the azimuth direction and a lattice point P2 located at the center between P1 and P3 as lattice points for preliminary measurement, at the shallowest position of the region-of-interest ROI.

The lattice point selector 66 supplies information on the selected lattice points P1 to P3 for preliminary measurement to the environmental sound velocity calculator 68.

In the illustrated example, three lattice points P1 to P3 are selected as the lattice points for preliminary measurement. However, the invention is not limited to this, and two points or four points or more may be selected.

Although the lattice points P1 to P3 at the shallowest position are selected as the lattice points for preliminary measurement, the invention is not limited to this, and lattice points at any depth may be selected as long as the positions of lattice points to be selected in the azimuth direction are different. It is preferable to select lattice points at the same depth as the lattice points for preliminary measurement.

In the illustrated example, in the azimuth direction, the lattice points P1 and P3 located at both ends of the region-of-interest ROI and the lattice point P2 located at the center between P1 and P3 are selected as the lattice points for preliminary measurement. However, the invention is not limited this, and lattice points at any position may be selected. For example, as shown in FIG. 4B, lattice points P1 and P3 located outside the region-of-interest ROI and a lattice point P2 located at the center between P1 and P3 may be selected as the lattice points for preliminary measurement.

The environmental sound velocity calculator 68 calculates environmental sound velocity values at the lattice points P1 to P3 for preliminary measurement.

Here, the environmental sound velocity value is sound velocity value where the contrast or the sharpness of an image becomes the highest when focus calculation is performed on respective lattice points on the basis of a set sound velocity so as to form an ultrasound image, and the set sound velocity is variously changed. For example, as described in JP 8-317926 A, the decision of the environmental sound velocity value can be performed on the basis of the contrast of an image, the spatial frequency in the scanning direction, variance, or the like.

The environmental sound velocity calculator 68 supplies respective environmental sound velocity values calculated regarding lattice points P1 to P3 for preliminary measurement to the environmental sound velocity comparator 70.

The environmental sound velocity comparator 70 compares the environmental sound velocity values of the lattice points P1 to P3 for preliminary measurement obtained by the environmental sound velocity calculator 68, and decides the presence or the absence of the measurement error due to the influence of refraction.

The environmental sound velocity comparator 70 obtains a difference Dv between the maximum value and the minimum value in the environmental sound velocity values of the lattice points P1, P2, and P3 for preliminary measurement, and compares the difference with a predetermined threshold.

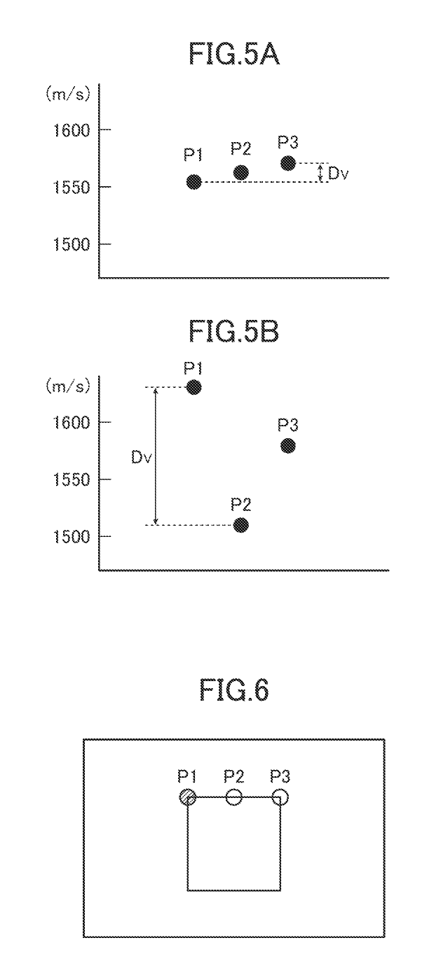

FIGS. 5A and 5B are measurement examples of the environmental sound velocity values of the lattice points P1, P2, and P3 for preliminary measurement. The predetermined threshold is 100 m/s in the example illustrated in FIG. 5.

As shown in FIG. 5A, when the difference Dv between the maximum value and the minimum value in the environmental sound velocity values (difference between P1 and P3 in the illustrated example) is equal to or smaller than the predetermined threshold, it is decided that there is no measurement error due to the influence of refraction, and a signal representing the decision is supplied to the main measuring unit 64 and the controller 36. In this case, the main measuring unit 64 measures the local sound velocity values of the set respective lattice points X_{ROI} (performs main sound velocity measurement).

On the other hand, as shown in FIG. 5B, when the difference Dv between the maximum value and the minimum value in the environmental sound velocity values (difference between P1 and P2 in the illustrated example) is greater than the predetermined threshold, it is determined that there is a measurement error due to the influence of refraction, and a signal representing the decision is supplied to the main measuring unit 64 and the controller 36. In this case, the main measuring unit 64 does not perform the measurement of the local sound velocity values (does not perform main sound velocity measurement).

As such, the environmental sound velocity values of lattice points that are fewer than lattice points to be used in the main sound velocity measurement are measured as preliminary measurement before the measurement of the local sound velocity values of the set lattice points X_{ROI} (main sound velocity measurement), and the presence or the absence of a measurement error caused by refraction is decided. Then, when it is decided that there is no measurement error, the main sound velocity measurement is performed, and when it is decided that there is a measurement error, the main sound velocity measurement is not performed. Therefore, the presence or the absence of influence of refraction can be quickly grasped, and the decision can be made before the main sound velocity measurement is performed even when there is a measurement error and re-measurement is required thereby shortening the time taken for measurement and calculation. Additionally, since the presence or the absence of the influence of refraction can be decided, when the main sound velocity measurement is performed, measurement or calculation with a small error can be performed thereby obtaining accurate local sound velocity values.

In addition, in the preliminary sound velocity measurement, the presence or the absence of irregularity of the sound velocity values in the azimuth direction due to the refraction of sound waves can be appropriately decided by comparing the environmental sound velocity values of lattice points at different positions in the azimuth direction.

In the illustrated example, the predetermined threshold is 100 m/s. However, the invention is not limited to this, and any threshold value may be used so long as the presence or the absence of a measurement error due to the influence of refraction can be appropriately decided. The predetermined threshold may be suitably determined according to the configuration of the apparatus, the performance required for the apparatus, or the like.

When the difference Dv between the maximum value and the minimum value in the environmental sound velocity values is greater than the predetermined threshold, it is preferable to display the results of the preliminary measurement so that a lattice point where the environmental sound velocity value is the maximum can be discriminated as illustrated in FIG. 6.

The main measuring unit 64 calculates the local sound velocity values in the set lattice points X_{ROI} when the preliminary measuring unit 62 decides that there is no measurement error due to the influence of refraction.

The main measuring unit 64 has an environmental sound velocity calculator 72 and a local sound velocity calculator 74.

The environmental sound velocity calculator 72 calculates environmental sound velocity values in the respective lattice points X_{ROI}. The method of calculating environmental sound velocity value in the environmental sound velocity calculator 72, is similar to that in the environmental sound velocity calculator 68. That is, sound velocity value where the contrast or the sharpness of an image becomes the highest when focus calculation is performed on respective lattice points on the basis of a set sound velocity so as to form an ultrasound image, and the set sound velocity is variously changed is obtained as the environmental sound velocity value.

The environmental sound velocity calculator 72 supplies the obtained environmental sound velocity values in the respective lattice points X_{ROI} to the local sound velocity calculator 74.

The local sound velocity calculator 74 calculates local sound velocity values in the respective lattice points X_{ROI}.

The method for calculating local sound velocity value that the local sound velocity calculator 74 performs is not particularly limited. For example, the calculation of the local sound velocity value can be performed by the method described in JP 2010-99452 A filed by the applicant of the present application.

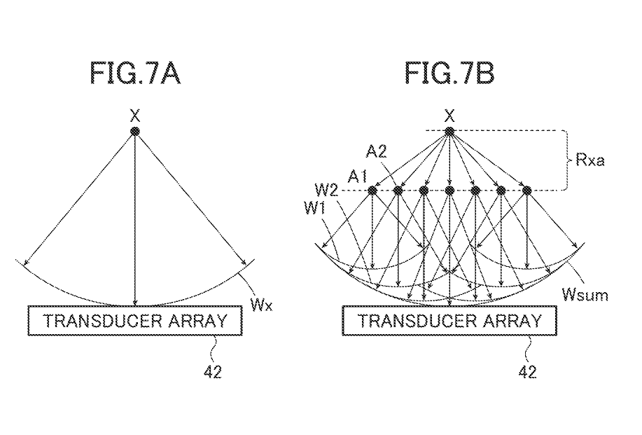

In the above-mentioned method, a local sound velocity value in a lattice point X is calculated using the fact that when an ultrasonic wave is transmitted into a subject and attention is paid to a reception wave Wx that reaches the transducer array 42 from the lattice point X that is a reflection point of the subject, as illustrated in FIG. 7A, and when a plurality of lattice points arranged at equal intervals at a position shallower than the lattice point X, i.e., a position close to the transducer array 42 are lattice points A1, A2, . . . , as illustrated in FIG. 7B, a synthetic wave Wsum of respective reception waves W1, W2, . . . from a plurality of lattice points A1, A2, . . . , that have received a reception wave from the lattice point X coincides with the reception wave Wx from the lattice point X according to the Huygens' principle.

First, environmental sound velocity values for all the lattice points X, A1, A2, . . . are acquired from the environmental sound velocity calculator 72.

Next, the waveform of the imaginary reception wave Wx radiated from the lattice point X is calculated using the environmental sound velocity value for the lattice point X.

Moreover, a hypothetical local sound velocity value V in the lattice point X is changed variously, and the imaginary synthetic wave Wsum of the reception waves W1, W2, . . . from the respective lattice points A1, A2, . . . is calculated. At this time, it is assumed that the sound velocity in a region Rxa between the lattice point X and the respective lattice points A1, A2 . . . is uniform and is equal to the local sound velocity value V at the lattice point X. The time periods until an ultrasonic wave propagated from the lattice point X reaches the lattice points A1, A2, . . . are XA1/V, XA2/V, . . . . Here, XA1, XA2, . . . are the distances between the lattice points A1, A2, . . . , and the lattice point X, respectively. Thus, the imaginary synthetic wave Wsum can be obtained by synthesizing reflected waves radiated from the lattice points A1, A2, . . . , with delay by the time periods XA1/V, XA2/V, . . . , respectively.

Next, the errors between a plurality of imaginary synthetic waves Wsum calculated by variously changing the hypothetical local sound velocity value V in the lattice point X and the imaginary reception wave Wx from the lattice point X are calculated, respectively, and the hypothetical local sound velocity value V where the error is minimum is decided to be the local sound velocity value in the lattice point X. Here, as the method for calculating the error between the imaginary synthetic wave Wsum and the imaginary reception wave Wx from the lattice point X, a method of taking a mutual correlation, a method of multiplying the reception wave Wx by the delay obtained from the synthetic wave Wsum to perform phase matching and addition, a method of multiplying the synthetic wave Wsum by the delay obtained from the reception wave Wx to perform phase matching and addition, and the like can be employed.

The local sound velocity values in the respective lattice points X_{ROI} within the region-of-interest ROI can be calculated as described above.

The local sound velocity calculator 74 associates the local sound velocity values in the respective lattice points X_{ROI} with position information on the respective lattice points X_{ROI} to produce a sound velocity map, and supplies the sound velocity map to the DSC 48 of the image producer 18.

The controller 36 controls respective parts of the ultrasound diagnostic apparatus on the basis of instructions input from the operation unit 38 by an operator.

The operation unit 38 is provided to allow the operator to perform input operation, and can be formed from a keyboard, a mouse, a trackball, a touch panel, or the like.

The storage unit 40 stores an operation program or the like, and recording media, such as a hard disk, a flexible disk, an MO, an MT, a RAM, a CD-ROM, and a DVD-ROM, can be used.

The signal processor 46, the DSC 48, the image processor 50, the display controller 32, and the sound-velocity calculator 24 are constituted by a CPU and operation programs for making the CPU perform various kinds of processing. However, these processors and controllers may be constituted by a digital circuit.

The ultrasound diagnostic apparatus 10 may have a plurality of display modes, and may have a configuration in which a desired image is displayed on the display unit 34 by selecting the display modes. For example, the ultrasound diagnostic apparatus may have a mode in which an ultrasound image (B-mode image) is displayed independently and a mode in which local sound velocity values (sound velocity map) are superimposed and displayed on a B-mode image (for example, the display of changing color or luminance according to a local sound velocity value or the display of connecting points where the local sound velocity values are equal to each other with a line), and may have a configuration in which an operator selects any one of the display modes from the operation unit 38.

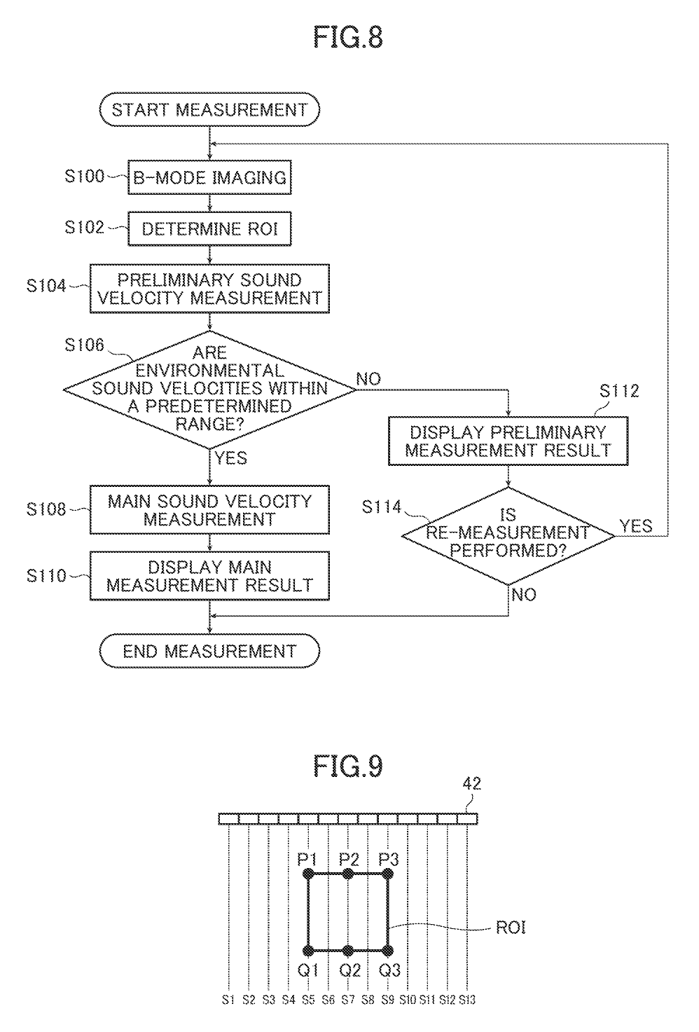

Next, the operation of the ultrasound diagnostic apparatus 10 will be described with reference to the flowchart of FIG. 8.

If an operator brings the ultrasound probe 12 into contact with the surface of a subject and starts measurement, ultrasonic beams are transmitted from the transducer array 42 according to actuation signals supplied from the transmission circuit 14, and the transducer array 42 receives ultrasonic echoes from the subject and outputs reception signals.

The reception circuit 16 produces reception data from the reception signals and supplies the reception data to the image producer 18. The signal processor 46 of the image producer 18 processes the reception data to produce B-mode image signals. The DSC 48 raster-converts the B-mode image signals, and the image processor 50 performs image processing to produce an ultrasound image. The produced ultrasound image is stored in the image memory 52 and also displayed on the display unit 34 by the display controller 32 (S100).

Next, the operator operates the operation unit 38 to input a setting instruction for the region-of-interest ROI, with reference to the displayed ultrasound image. According to the instruction from the operation unit 38, the region-of-interest setter 60 sets a region-of-interest ROI and also sets a plurality of lattice points X_{ROI} that are arranged two-dimensionally (S102).

When the region-of-interest ROI and the lattice points X_{ROI} are set, the preliminary measuring unit 62 measures environmental sound velocity values regarding several lattice points for preliminary measurement that are fewer than lattice points used for main sound velocity measurement (S104), and decides whether or not the difference Dv between the maximum value and the minimum value in the environmental sound velocity values of the respective lattice points for preliminary measurement is equal to or lower than the predetermined threshold (S106).

When the difference Dv between the maximum value and the minimum value in the environmental sound velocity values of the lattice points for preliminary measurement is equal to or lower than the predetermined threshold, the main measuring unit 64 calculates the local sound velocity values of the respective lattice points X_{ROI} (S108), displays the calculation results on the display unit 34 as a sound velocity map (S110), and ends the measurement.

On the other hand, when the difference Dv between the maximum value and the minimum value in the environmental sound velocity values of the lattice points for preliminary measurement is higher than the predetermined threshold, a notice showing the presence of measurement error is displayed on the display unit 34 as a result of the preliminary measurement (S112), and a notice as to whether re-measurement is requested or not is displayed on the display unit 34 (S114). When re-measurement is performed, it is started from the imaging of a B-mode image (S100). On the other hand, when re-measurement is not performed, the measurement is ended.

As mentioned above, the ultrasound diagnostic apparatus 10 that implements the ultrasound image producing method of the invention measures the environmental sound velocity values of lattice points which have different positions in the azimuth direction and which are fewer than the lattice points to be used in the main sound velocity measurement, as preliminary measurement before the measurement (main sound velocity measurement) of the local sound velocity values of the set lattice points X_{ROI}, and decides the presence or the absence of a measurement error due to the influence of refraction. Then, when it is decided that there is no measurement error, the main sound velocity measurement is performed, and when it is decided that there is a measurement error, the main sound velocity measurement is not performed. Thus, the presence or the absence of the influence of refraction can be quickly grasped, and the decision can be made before the main sound velocity measurement is performed even when there is a measurement error and re-measurement is required. Accordingly, the time taken for measurement and calculation can be shortened. Moreover, since the presence or the absence of the influence of refraction can be decided, when the main sound velocity measurement is performed, measurement or calculation with a small error can be performed thereby obtaining accurate local sound velocity values.

In the illustrated example, the preliminary measuring unit 62 selects the lattice points P1 to P3 for preliminary measurement in one row of the same depth, and performs comparison of the environmental sound velocity values to decide the presence or the absence of the influence of refraction. However, the invention is not limited to this, and lattice points for preliminary measurement may be selected in two or more rows of different depths, and the comparison of environmental sound velocity values may be performed in the respective rows.

FIG. 9 is a view conceptually illustrating lattice points for preliminary measurement.

As shown in FIG. 9, the lattice point selector 66 selects lattice points P1 and P3 located at both ends of the region-of-interest ROI in the azimuth direction and a lattice point P2 located at the center between P1 and P3 as lattice points for preliminary measurement, at the shallowest position of the region-of-interest ROI, and selects lattice points Q1 and Q3 located at both ends of the region-of-interest ROI in the azimuth direction and a lattice point Q2 located at the center between Q1 and Q3 as lattice points for preliminary measurement, at the deepest position of the region-of-interest ROI.

When the lattice points for preliminary measurement are selected at a plurality of depths in this way, the environmental sound velocity comparator 70 obtains the difference between the maximum value and the minimum value in the environmental sound velocity values in the lattice points P1 to P3 for preliminary measurement and performs comparison of the difference with the predetermined threshold, and also obtains the difference between the maximum value and the minimum value in the environmental sound velocity values in the lattice points Q1 to Q3 for preliminary measurement and performs comparison of the difference with the predetermined threshold. As a result of the comparison, when both of the differences are equal to or lower than the predetermined threshold, the main measuring unit 64 may calculate the local sound velocity values of the respective lattice points X_{ROI} and may obtain a sound velocity map.

In the above example, the configuration in which transmission and reception of ultrasonic waves regarding one tomographic plane are performed using a one-dimensional transducer array as the transducer array has been adopted. However, the invention is not limited to this, and a configuration in which transmission and reception of ultrasonic waves regarding a plurality of tomographic planes are performed using a two-dimensional transducer array may be adopted.

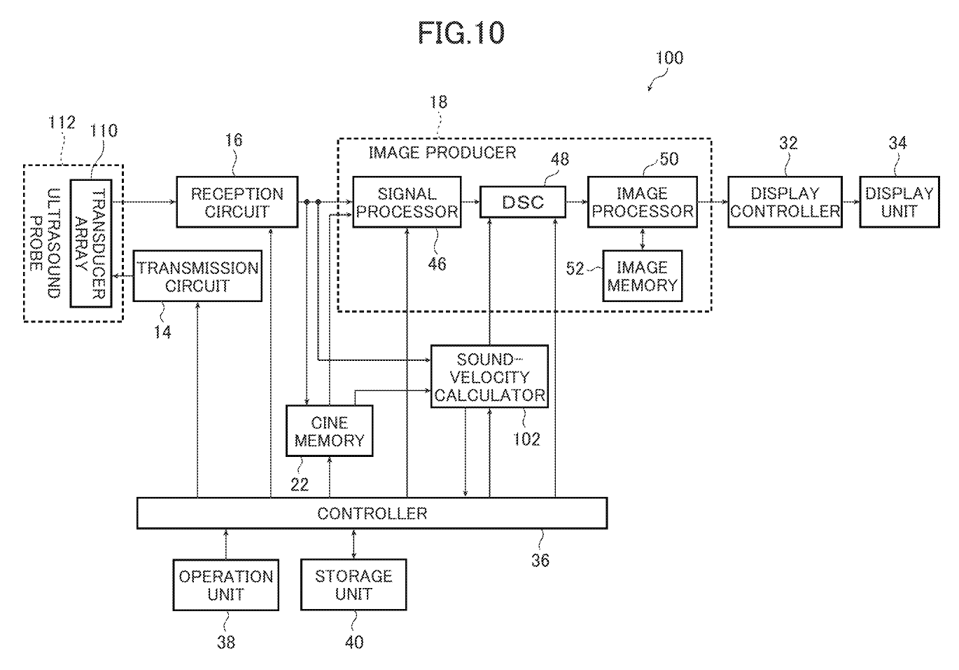

FIG. 10 is a block diagram conceptually illustrating the configuration of another example of the ultrasound diagnostic apparatus that implements the ultrasound image producing method of the invention. Since the ultrasound diagnostic apparatus 100 illustrated in FIG. 10 basically has the same configuration as the ultrasound diagnostic apparatus 10 illustrated in FIG. 1 except that the ultrasound diagnostic apparatus 100 has a transducer array 110 instead of the transducer array 42 and has a sound-velocity calculator 102 instead of the sound-velocity calculator 24, the same parts will be designated by the same reference numerals, and the following description will mainly be made about the different parts.

The ultrasound diagnostic apparatus 100 has an ultrasound probe 112 having a transducer array 110, the transmission circuit 14 and the reception circuit 16 that are connected to the transducer array 110, the image producer 18, the cine memory 22, the sound-velocity calculator 102, the display controller 32, the display unit 34, the controller 36, the operation unit 38, and the storage unit 40.

The ultrasound probe 112 has a two-dimensional transducer array 110.

The transducer array 110 has a plurality of ultrasound transducers that are arranged two-dimensionally. In the imaging of an ultrasound image, the ultrasound transducers transmit ultrasonic beams according to actuation signals supplied from the transmission circuit 14 and receives ultrasonic echoes from a subject to output reception signals.

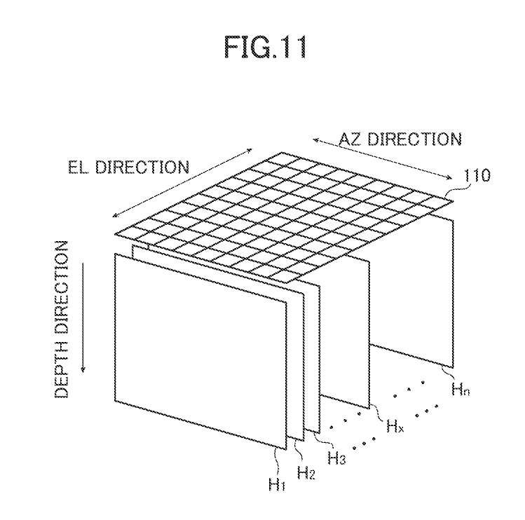

FIG. 11 schematically illustrates the two-dimensional transducer array 110, and tomographic planes H₁, H₂, H₃, . . . , Hx, . . . , and Hn for which the two-dimensional transducer array 110 performs transmission and reception of ultrasonic waves to acquire information on a subject.

As shown in FIG. 11, the tomographic planes are planes parallel to the scanning direction (the AZ direction) of ultrasonic waves according to the arrangement of the ultrasound transducers of the transducer array 110, and a plurality of tomographic planes are set in a direction (the direction of EL) perpendicular to the AZ direction and the depth direction.

In the present example, transmission and reception of ultrasonic waves can be performed in each tomographic plane to perform imaging of an ultrasound image and measurement of sound velocity values (local sound velocity values and environmental sound velocity values).

Each of the ultrasound transducers in the transducer array 110 comprises a vibrator composed of a piezoelectric body and electrodes each provided on both ends of the piezoelectric body. The piezoelectric body is composed of, for example, a piezoelectric ceramic represented by a PZT (lead zirconate titanate), a polymeric piezoelectric device represented by PVDF (polyvinylidene fluoride), or a piezoelectric monocrystal represented by PMN-PT (lead magnesium niobate lead titanate solid solution).

If a pulsed or continuous-wave voltage is applied to the electrodes of the vibrators, the piezoelectric substances expand and contract, pulsed or continuous-wave ultrasonic waves are generated from the respective vibrators, and an ultrasonic beam is formed by the synthesis of the ultrasonic waves. If the respective vibrators receive propagated ultrasonic waves, they expand and contract to generate electrical signals, and the electrical signals are output as reception signals of the ultrasonic waves.

The transmission circuit 14 supplies actuation signals to the plurality of ultrasound transducers on the basis of a selected transmission delay pattern similarly to the above description.

The reception circuit 16, similarly to the above description, amplifies and A/D converts reception signals transmitted from the respective ultrasound transducers, and then performs the reception focus processing to produce reception data (sound ray signals).

The reception circuit 16 supplies the reception data to the image producer 18, the cine memory 22, and the sound-velocity calculator 24.

The image producer 18, similarly to the above description, produces an ultrasound image from the reception data supplied from the reception circuit 16.

The image producer 18 has the signal processor 46, the DSC 48, the image processor 50, and the image memory 52.

The signal processor 46, similarly to the above description, produces B-mode image signals from the reception data produced in the reception circuit 16.

The DSC (Digital Scan Converter) 48, similarly to the above description, raster-converts the produced B-mode image signals and the signals of the sound velocity map.

The image processor 50, similarly to the above description, performs various kinds of required image processing on the B-mode image signals input from the DSC 48, and then, outputs the processed B-mode image signals to the display controller 32 or stores these signals in the image memory 52.

The display controller 32, similarly to the above description, makes an ultrasound diagnostic image display on the display unit 34 on the basis of the B-mode image signals subjected to the image processing by the image processor 50.

The cine memory 22, similarly to the above description, sequentially stores the reception data output from the reception circuit 16.

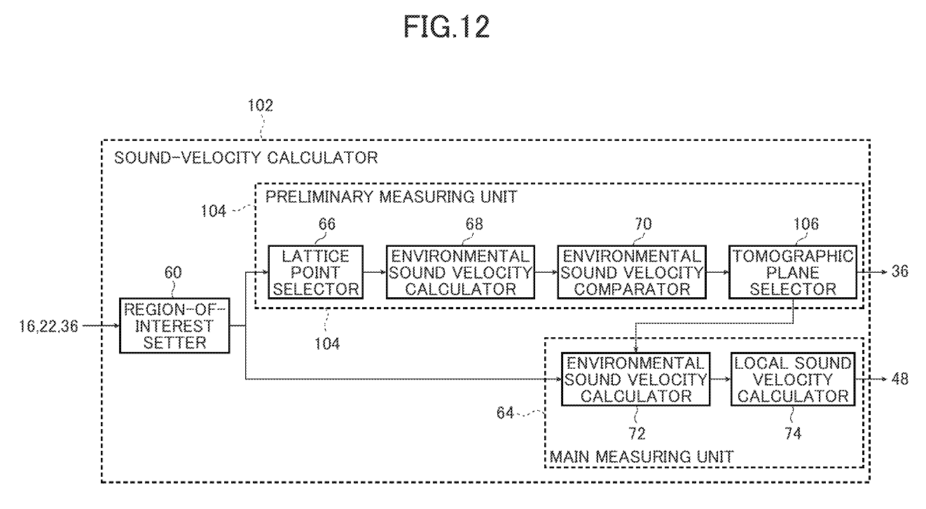

FIG. 12 is a block diagram conceptually illustrating the configuration of the sound-velocity calculator 102.

The sound-velocity calculator 102 calculates local sound velocity values in tissue to be diagnosed in a subject and produces a sound velocity map showing sound velocity values and position information, under the control of the controller 36.

Here, in the invention, the sound-velocity calculator 102 performs preliminary sound velocity measurement that measures environmental sound velocity values to decide the presence or the absence of a measurement error caused by refraction, and then, performs the measurement (main sound velocity measurement) of the local sound velocity values.

The sound-velocity calculator 102 has the region-of-interest setter 60, the preliminary measuring unit 104, and the main measuring unit 64.

As mentioned above, the region-of-interest setter 60 sets a region-of-interest ROI within a subject, sets a lattice two-dimensionally in the region-of-interest ROI, and sets the lattice points X_{RQI} subjected to the measurement.

In addition, in the present example, the region-of-interest setter 60 sets a region-of-interest ROI and lattice points X_{ROI} common to all the tomographic planes. As an example, the region-of-interest ROI and the lattice points X_{ROI} that are shown in FIG. 4 are set for each tomographic plane.

The region-of-interest setter 60 supplies information on the region-of-interest ROI and the plurality of lattice points X_{ROI} that are set, to the preliminary measuring unit 104 and the main measuring unit 64.

As preliminary sound velocity measurement before the measurement (main sound velocity measurement) of the local sound velocity values, the preliminary measuring unit 104 measures environmental sound velocity values regarding several points among the plurality of lattice points X_{ROI} set by the region-of-interest setter 60 and decides the presence or the absence of a measurement error caused by refraction, in each tomographic plane Hx.

The preliminary measuring unit 104 has the lattice point selector 66, the environmental sound velocity calculator 68, the environmental sound velocity comparator 70, and a tomographic plane selector 106.

As mentioned above, the lattice point selector 66 selects lattice points for preliminary measurement to be used for the preliminary sound velocity measurement from the plurality of lattice points X_{ROI} set by the region-of-interest setter 60.

In addition, the lattice point selector 66 selects lattice points for preliminary measurement common to each tomographic plane.

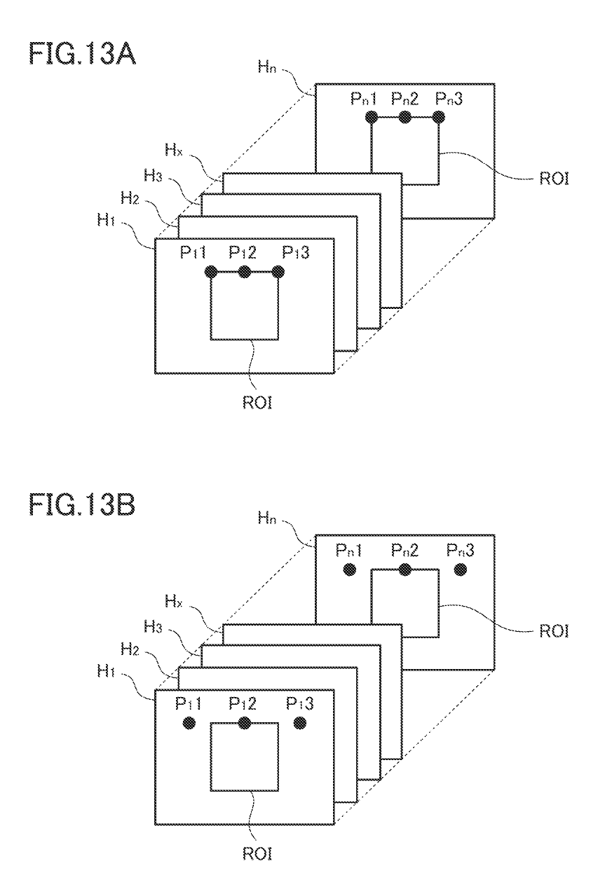

FIG. 13A is a view schematically illustrating selected lattice points for preliminary measurement.

As shown in FIG. 13A, in each tomographic plane Hx, the lattice point selector 66 selects lattice points Px1 and Px3 located at both ends of the region-of-interest ROI in the azimuth direction and a lattice point Px2 located at the center between Px1 and Px3 as lattice points for preliminary measurement to be used for the preliminary sound velocity measurement, at the shallowest position of the region-of-interest ROI.

The lattice point selector 66 supplies information on the selected lattice points Px1 to Px3 for preliminary measurement to the environmental sound velocity calculator 68.

In the illustrated example, in the azimuth direction, the lattice points Px1 and Px3 located at both ends of the region-of-interest ROI and the lattice point Px2 located at the center between Px1 and Px3 are selected as the lattice points for preliminary measurement. However, the invention is not limited this, and lattice points at any position may be selected. For example, as shown in FIG. 13B, lattice points Px1 and Px3 located outside the region-of-interest ROI and a lattice point Px2 located at the center between Px1 and Px3 may be selected as the lattice points for preliminary measurement.

As mentioned above, the environmental sound velocity calculator 68 calculates environmental sound velocity values in the lattice points for preliminary measurement. In the present example, the environmental sound velocity calculator 68 calculates environmental sound velocity values at the lattice points Px1 to Px3 for preliminary measurement in each tomographic plane Hx.

The environmental sound velocity calculator 68 supplies respective environmental sound velocity values calculated regarding lattice points Px1 to Px3 for preliminary measurement to the environmental sound velocity comparator 70.

As mentioned above, the environmental sound velocity comparator 70 compares the environmental sound velocity values of the lattice points Px1 to Px3 for preliminary measurement obtained by the environmental sound velocity calculator 68, and decides the presence or the absence of a measurement error due to the influence of refraction.

In the present example, the environmental sound velocity comparator 70 obtains a difference (measured sound velocity difference) DvX between the maximum value and the minimum value in the environmental sound velocity values of the lattice points Px1, Px2, and Px3 for preliminary measurement, and compares the difference with the predetermined threshold in every tomographic plane Hx.

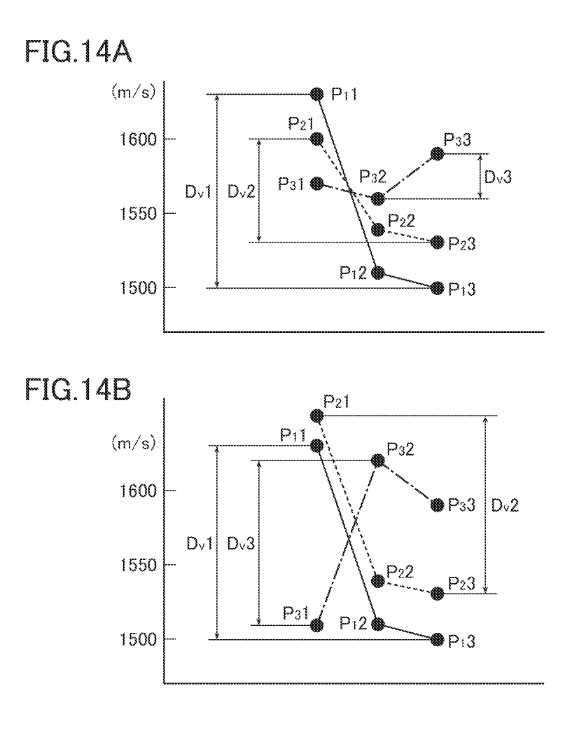

FIGS. 14A and 14B are measurement examples of the environmental sound velocity values of the lattice points Px1, Px2, and Px3 for preliminary measurement. FIG. 14 illustrates measurement examples of environmental sound velocity values regarding three tomographic planes for simplicity. The predetermined threshold is 100 m/s in the examples illustrated in FIG. 14.

In the example illustrated in FIG. 14A, the measured sound velocity difference Dv1 in the tomographic plane H₁ is about 160 m/s and is greater than the predetermined threshold, and the measured sound velocity differences Dv2 and Dv3 in the tomographic planes H₂ and H₃ are about 70 m/s and about 50 m/s, respectively, and are smaller than the predetermined threshold. In this case, in the tomographic plane H₁, it is determined that there is a measurement error due to the influence of refraction, and in the tomographic planes H₂ and H₃, it is determined that there is no measurement error due to the influence of refraction.

On the other hand, in the example illustrated in FIG. 14B, the measured sound velocity differences Dv1, Dv2, and Dv3 in the tomographic planes H₁, H₂, and H₃ are about 160 m/s, about 130 m/s, and about 110 m/s, respectively, and are greater than the predetermined threshold. In this case, in the tomographic planes H₁, H₂, and H₃, it is decided that there is a measurement error due to the influence of refraction.

The environmental sound velocity comparator 70 supplies the determination result and information on the measured sound velocity difference DvX to the tomographic plane selector 106.

The tomographic plane selector 106 selects a tomographic plane Hx where the main sound velocity measurement should be performed, on the basis of the information supplied from the environmental sound velocity comparator 70. Specifically, as for tomographic planes Hx for which it is decided that there is no measurement error due to the influence of refraction, the measured sound velocity differences DvX in the respective tomographic planes Hx are compared by the environmental sound velocity comparator 70, and a tomographic plane Hx having a smallest measured sound velocity difference DvX is selected as a tomographic plane where the main sound velocity measurement is performed.

For example, in the example illustrated in FIG. 14A, when the measured sound velocity differences Dv2 and Dv3 in tomographic planes H₂ and H₃ for which it is decided that there is no measurement error due to the influence of refraction are compared by the environmental sound velocity comparator 70, Dvc3 is smaller than Dv2. Thus, the tomographic plane H₃ is selected as a tomographic plane where the main sound velocity measurement is performed.

On the other hand, no tomographic plane is selected when the measured sound velocity differences DvX in all the tomographic planes are greater than the predetermined threshold as in the example illustrated in FIG. 14B.

The tomographic plane selector 106 supplies information on the selected tomographic plane or information to the effect that there is no suitable tomographic plane to the main measuring unit 64 and the controller 36. The main measuring unit 64 performs the main sound velocity measurement in the tomographic plane selected by the tomographic plane selector 106. The main measuring unit 64 does not perform the main sound velocity measurement when there is no suitable tomographic plane.

As mentioned above, in the configuration in which information on a plurality of two-dimensional tomographic planes can be acquired in the direction orthogonal to the tomographic planes using the two-dimensional transducer array, the environmental sound velocity values of lattice points that are fewer than lattice points to be used in the main sound velocity measurement, are measured in the plurality of tomographic planes, respectively, as preliminary sound velocity measurement before the measurement (main sound velocity measurement) of the local sound velocity values of the set lattice points X_{ROI}, and the presence or the absence of a measurement error due to the influence of refraction is decided. Then, the main sound velocity measurement is performed in tomographic planes decided that there is no measurement error, and when it is decided that there is a measurement error in all the tomographic planes, the main sound velocity measurement is not performed. Thus, the presence or the absence of the influence of refraction can be quickly grasped, and the decision can be made before the main sound velocity measurement is performed even when there is a measurement error and re-measurement is required. Accordingly, the time taken for measurement and calculation can be shortened. Moreover, since the presence or the absence of the influence of refraction is decided in respective tomographic planes, a tomographic plane for which it is decided that there is no measurement error is selected, and the main sound velocity measurement is performed for the selected tomographic plane, when the main sound velocity measurement is performed, measurement or calculation with a small error can be performed thereby obtaining accurate local sound velocity values.

In addition, in the preliminary sound velocity measurement, the presence or the absence of variations of sound velocity values in the azimuth direction due to the refraction of sound waves can be appropriately decided by comparing the environmental sound velocity values in lattice points at different positions in the azimuth direction.

In the illustrated example, the predetermined threshold is 100 m/s. However, the invention is not limited to this, and any desired threshold value may be used so long as the presence or the absence of a measurement error due to the influence of refraction can be appropriately decided. The predetermined threshold may be suitably determined according to the configuration of the apparatus, performance required for the apparatus, or the like.

When the measured sound velocity differences DvX are greater than the predetermined threshold in all the tomographic planes, it is preferable that a lattice point with the highest frequency, where an environmental sound velocity value is a maximum value, be allowed to discriminate, so as to display the results of the preliminary sound velocity measurement.

In the illustrated example, a tomographic plane Hx where a measured sound velocity difference DvX is the smallest is selected as a tomographic plane where the main sound velocity measurement should be performed. However, the invention is not limited to this, and any tomographic plane Hx may be selected as long as a tomographic plane Hx where a measured sound velocity difference DvX is smaller than the predetermined threshold.

In addition, it is preferable that the tomographic plane Hx where the measured sound velocity difference DvX is the smallest be selected as a tomographic plane where the main sound velocity measurement is performed. Thereby, a tomographic plane where a measurement error due to the influence of refraction is smaller can be selected.

In the illustrated example, the measured sound velocity differences DvX in all the tomographic planes Hx are obtained to decide the presence or the absence of a measurement error in each tomographic planes Hx, and then, a tomographic plane Hx for the main sound velocity measurement is selected from the tomographic planes Hx for which it is decided that there is no measurement error. However the invention is not limited to this. In the respective tomographic planes Hx, the measurement of the environmental sound velocity values, the calculation of the measured sound velocity differences DvX, and the comparison between the measured sound velocity differences DvX and the predetermined threshold may be performed sequentially, and a tomographic plane Hx where the measured sound velocity differences DvX become equal to or lower than the predetermined threshold first may be selected as a tomographic plane Hx for the main sound velocity measurement.

The configuration in which the measured sound velocity differences DvX are obtained in all the tomographic planes Hx to decide the presence or the absence of a measurement error, and then a tomographic plane Hx for the main sound velocity measurement is selected is preferable in that a tomographic plane where a measurement error due to the influence of refraction is smaller can be selected. On the other hand, the configuration in which the presence or the absence of a measurement error is performed sequentially, and a tomographic plane Hx where the measured sound velocity differences DvX become equal to or lower than the predetermined threshold first is selected as a tomographic plane Hx for the main sound velocity measurement is preferable in that operation time can be reduced.

As mentioned above, the main measuring unit 64 calculates local sound velocity values in the set lattice points X_{ROI}. In the present example, the main measuring unit 64 calculates the local sound velocity values in a tomographic plane where the preliminary measuring unit 104 has decided that there is no measurement error due to the influence of refraction.

The main measuring unit 64 has the environmental sound velocity calculator 72 and the local sound velocity calculator 74.

The environmental sound velocity calculator 72, similarly to the above description, calculates environmental sound velocity values in the respective lattice points X_{ROI}.

The environmental sound velocity calculator 72 supplies the obtained environmental sound velocity values in the respective lattice points X_{ROI} to the local sound velocity calculator 74.

The local sound velocity calculator 74, similarly to the above description, calculates local sound velocity values in the respective lattice points X_{ROI}.

The local sound velocity calculator 74 associates the local sound velocity values in the respective lattice points X_{ROI} with position information on the respective lattice points X_{ROI} to produce a sound velocity map, and supplies the sound velocity map to the DSC 48 of the image producer 18. The information on the sound velocity map is converted into image signals by the DSC 48 and is displayed on the display unit 34.

The controller 36, similarly to the above description, controls respective parts of the ultrasound diagnostic apparatus on the basis of instructions input from the operation unit 38 by an operator.

The operation unit 38, similarly to the above description, is provided to allow the operator to perform input operation, and can be formed from a keyboard, a mouse, a trackball, a touch panel, or the like.

The storage unit 40, similarly to the above description, stores an operation program or the like.

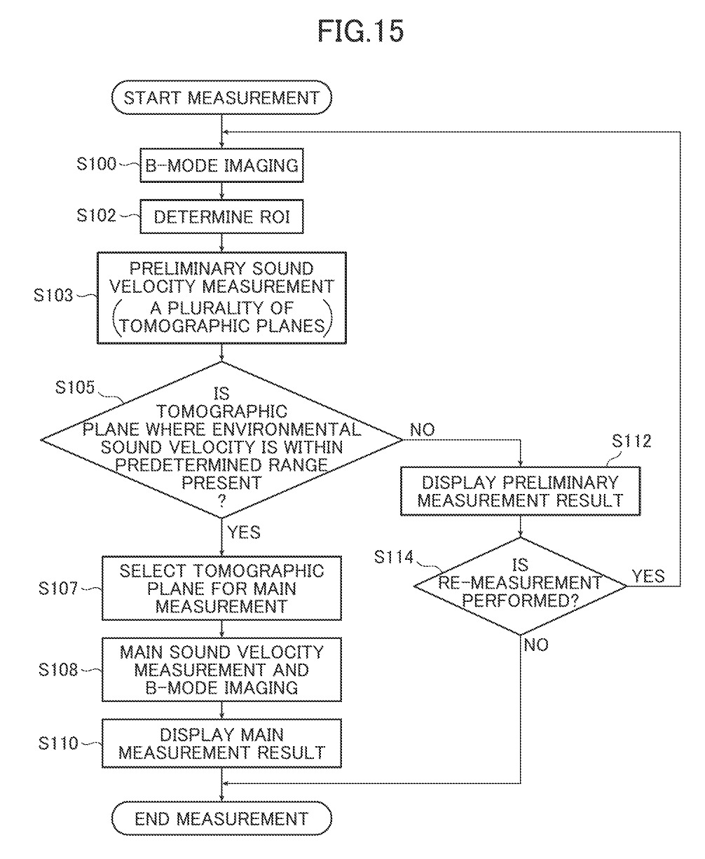

Next, the operation of the ultrasound diagnostic apparatus 10 will be described with reference to the flowchart of FIG. 15.

First, the imaging of a B-mode image is performed in an arbitrary tomographic plane.

Specifically, if an operator brings the ultrasound probe 112 into contact with the surface of a subject and starts measurement, in an arbitrary tomographic plane, ultrasonic beams are transmitted from the transducer array 42 according to actuation signals supplied from the transmission circuit 14, and the transducer array 42 receives ultrasonic echoes from a subject and outputs reception signals.

The reception circuit 16 produces reception data from the reception signals and supplies the reception data to the image producer 18. The signal processor 46 of the image producer 18 processes the reception data to produce B-mode image signals. The DSC 48 raster-converts the B-mode image signals, and the image processor 50 performs image processing to produce an ultrasound image. The produced ultrasound image is stored in the image memory 52 and also displayed on the display unit 34 by the display controller 32 (S100).

Next, the operator operates the operation unit 38 to input a setting instruction for the region-of-interest ROI, with reference to the displayed ultrasound image. According to the instruction from the operation unit 38, the region-of-interest setter 60 sets a region-of-interest ROI in all the tomographic planes Hx and also sets a plurality of lattice points X_{ROI} that are arranged two-dimensionally (S102).

When the region-of-interest ROI and the lattice points X_{ROI} are set, the preliminary measuring unit 104 measures environmental sound velocity values regarding several lattice points for preliminary measurement that are fewer than lattice points used for main sound velocity measurement in all the tomographic planes Hx (S103), and decides whether or not the measured sound velocity difference DvX is equal to or lower than the predetermined threshold in each tomographic plane Hx (S105).

When there are tomographic planes Hx where the measured sound velocity differences DvX are equal to or lower than the predetermined threshold, a tomographic plane Hx used for the main sound velocity measurement is selected from the tomographic planes Hx where the measured sound velocity differences DvX are equal to or lower than the predetermined threshold (S107). When the tomographic plane Hx for the main sound velocity measurement is selected, imaging of a B-mode image (ultrasound image) is performed in the selected tomographic plane Hx, and the main measuring unit 64 performs the main sound velocity measurement in the selected tomographic plane Hx (S108), display the ultrasound image and the measurement results of the sound velocity values on the display unit 34 (S110), and ends the measurement.

On the other hand, when the measured sound velocity difference DvX is higher than the predetermined threshold in all the tomographic planes, a notice showing the presence of measurement error is displayed on the monitor 34 as a result of the preliminary sound velocity measurement (S112), and a notice as to whether re-measurement is requested or not is displayed on the display unit 34 (S114). When re-measurement is performed, it is started from the imaging of a B-mode image used for setting of ROI (S100). On the other hand, when re-measurement is not performed, the measurement is ended.

As mentioned above, in the configuration in which information on a plurality of two-dimensional tomographic planes can be acquired in the direction orthogonal to the tomographic planes using the two-dimensional transducer array, the ultrasound diagnostic apparatus 100 that implements the ultrasound image producing method of the invention measures the environmental sound velocity values of lattice points which are fewer than lattice points to be used in the main sound velocity measurement and have different positions in the azimuth direction, in the plurality of tomographic planes, respectively, as preliminary sound velocity measurement before the measurement (main sound velocity measurement) of the local sound velocity values of the set lattice points X_{ROI}, and decides the presence or the absence of a measurement error due to the influence of refraction. Then, the main sound velocity measurement is performed in tomographic planes for which it is decided that there is no measurement error, and when it is decided that there is a measurement error in all the tomographic planes, the main sound velocity measurement is not performed. Thus, the presence or absence of the influence of refraction can be quickly grasped, and the decision can be made before the main sound velocity measurement is performed even when there is a measurement error and re-measurement is required. Accordingly, the time taken for measurement and calculation can be shortened. Moreover, since the presence or the absence of the influence of refraction is decided in respective tomographic planes, a tomographic plane for which it is decided that there is no measurement error is selected, and the main sound velocity measurement is performed for the selected tomographic plane, when the main sound velocity measurement is performed, measurement or calculation with a small error can be performed thereby obtaining accurate local sound velocity values.

In the illustrated example, the preliminary measuring unit 104 selects the lattice points P1 to P3 for preliminary measurement in one row of the same depth, and performs comparison of the environmental sound velocity values to decide the presence or the absence of the influence of refraction. However, the invention is not limited to this, and lattice points for preliminary measurement may be selected in two or more rows of different depths, and comparison of environmental sound velocity values may be performed in the respective rows.

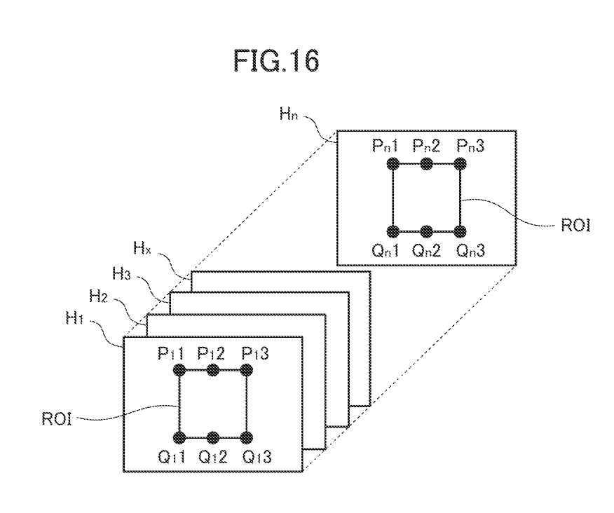

FIG. 16 is a view conceptually illustrating lattice points for preliminary measurement.

As shown in FIG. 16, the lattice point selector 66 selects lattice points P1 and P3 located at both ends of the region-of-interest ROI in the azimuth direction and a lattice point P2 located at the center between P1 and P3 as lattice points for preliminary measurement, at the shallowest position of the region-of-interest ROI, and selects lattice points Q1 and Q3 located at both ends of the region-of-interest ROI in the azimuth direction and a lattice point Q2 located at the center between Q1 and Q3 as lattice points for preliminary measurement, at the deepest position of the region-of-interest ROI.

When the lattice points for preliminary measurement are selected at a plurality of depths in this way, the environmental sound velocity comparator 70 obtains the difference between the maximum value and the minimum value in the environmental sound velocity values in the lattice points P1 to P3 for preliminary measurement in each tomographic plane and performs comparison of the difference with a predetermined threshold, and also obtains the difference between the maximum value and the minimum value in the environmental sound velocity values in the lattice points Q1 to Q3 for preliminary measurement and performs comparison of the difference with the predetermined threshold. As result of the comparison, when both of the differences are equal to or lower than the predetermined threshold, it is decided to be a tomographic plane having no measurement error due to the influence of refraction.

The invention is basically as above.

Although the invention has been described above in detail, the invention is not limited to the above embodiment, and various improvements and modifications may be made without departing from the scope of the invention.

What is claimed is:

1. An ultrasound image producing method comprising:
an image producing step for transmitting ultrasonic waves and receiving ultrasonic echoes reflected by a subject to output reception signals according to the received ultrasonic waves, by a transducer array, and performing signal processing of the reception signals to produce a B-mode image by an image producer;
a lattice setting step for setting a region-of-interest on the produced B-mode image and setting a lattice on the set region-of-interest;
a preliminary sound velocity measuring step for measuring environmental sound velocity values of two or more lattice points of the lattice located at different positions in a scanning direction of the ultrasonic waves;
a detecting step for detecting whether or not a measured sound velocity difference that is a difference between a maximum value and a minimum value in environmental sound velocity values measured in the preliminary sound velocity measuring step is equal to or lower than a predetermined threshold; and
a main sound velocity measuring step for calculating local sound velocity values in lattice points of the lattice when the measured sound velocity difference is equal to or lower than the predetermined threshold.

2. The ultrasound image producing method according to claim 1,
wherein environmental sound velocity values of two or more lattice points in the scanning direction of the ultrasonic waves are further measured at different depths in the preliminary sound velocity measuring step, and
wherein whether or not measured sound velocity differences are equal to or lower than predetermined threshold at all the depths is detected in the detecting step, and the main sound velocity measuring step is performed when the measured sound velocity differences are equal to or lower than the predetermined threshold at all the depths.

3. The ultrasound image producing method according to claim 1,
wherein a lattice with a size that exceeds the set region-of-interest is set in the lattice setting step.

4. The ultrasound image producing method according to claim 1,
wherein a number of lattice points where environmental sound velocity values are to be measured in the preliminary sound velocity measuring step is smaller than a number of lattice points where local sound velocity values are to be calculated in the main sound velocity measuring step.

5. The ultrasound image producing method according to claim 1, further comprising a step for comparing the environmental sound velocity values measured in the preliminary sound velocity measuring step and displaying a lattice point showing a largest environmental sound velocity value in a discriminative manner.

6. The ultrasound image producing method according to claim 1, further comprising a step for displaying notice or measurement results of environmental sound velocity values when the difference between the maximum value and the minimum value in the environmental sound velocity values exceeds the predetermined threshold in the detecting step.

7. The ultrasound image producing method according to claim 1,
wherein measurement results of the local sound velocity values in the main sound velocity measuring step are superimposed and displayed on the B-mode image.

8. The ultrasound image producing method according to claim 1,
wherein the transducer array is a two-dimensional transducer array having transducers that are arranged two-dimensionally, and acquiring a plurality of information on two-dimensional tomographic planes in a direction orthogonal to the tomographic planes, according to an arrangement of the transducers,
wherein the preliminary sound velocity measuring step and the detecting step are performed in each of the plurality of tomographic planes, and
wherein the main sound velocity measuring step is performed in a tomographic plane where the measured sound velocity difference is equal to or lower than the predetermined threshold.

9. The ultrasound image producing method according to claim 8, further comprising a step for selecting a tomographic plane where the measured sound velocity difference is a smallest,
wherein the main sound velocity measuring step is performed in the selected tomographic plane.

10. The ultrasound image producing method according to claim 8,
wherein the preliminary sound velocity measuring step and the detecting step are performed sequentially in the plurality of tomographic planes, and the main sound velocity measuring step is performed in a tomographic plane where the measured sound velocity difference becomes equal to or lower than the predetermined threshold first.

11. An ultrasound image diagnostic apparatus comprising:
a transducer array that transmits ultrasonic waves and receives ultrasonic echoes reflected by a subject to output reception signals according to the received ultrasonic waves;
an image producer that produces an ultrasound image based on the reception signals output from the transducer array;
a region-of-interest setter that sets a region-of-interest within an imaging region, sets a lattice on the set region-of-interest, and sets lattice points;
a preliminary sound velocity measuring unit that measures environmental sound velocity values of two or more lattice points of the lattice located at different positions in a scanning direction of the ultrasonic waves, and that calculates a measured sound velocity difference that is a difference between a maximum value and a minimum value in the measured environmental sound velocity values; and
a main sound velocity measuring unit that calculates local sound velocity values in lattice points of the lattice when the measured sound velocity difference calculated by the preliminary sound velocity measuring unit is equal to or lower than the predetermined value.

12. The ultrasound image diagnostic apparatus according to claim 11,
wherein the transducer array is a two-dimensional transducer array having transducers that are arranged two-dimensionally, and acquiring a plurality of information on two-dimensional tomographic planes in a direction orthogonal to the tomographic planes, according to an arrangement direction of the transducers,
wherein the preliminary sound velocity measuring unit calculates the measured sound velocity differences in each of the tomographic planes, and
wherein the main sound velocity measuring unit calculates local sound velocity values in lattice points of the lattice, in a tomographic plane where the measured sound velocity difference calculated by the preliminary sound velocity measuring unit is equal to or lower than the predetermined threshold.
